(19) United States
(12) Patent Application Publication
(10) Pub. No.: US 2025/0101506 A1
(43) Pub. Date: Mar. 27, 2025

(54) PCR KIT-OF-PARTS, METHOD AND SYSTEM

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 18/291,403

(22) PCT Filed: Jul. 26, 2021
(86) PCT No.: PCT/US2021/043118

§ 371 Date: Jan. 23, 2024

(51) Int. Cl. C12Q 1/686; B01L 3/00; B01L 7/00; C12Q 1/6823

(52) U.S. Cl. CPC C12Q 1/686; B01L 3/502715; B01L 7/52; C12Q 1/6823; B01L 2300/0663; B01L 2300/0816; B01L 2300/18

(57) ABSTRACT

A PCR kit-of-parts is described. The kit-of-parts comprises a first oligonucleotide bound to a bead by a cleavable linker; a second oligonucleotide bound to a bead by a cleavable linker; and an enzyme bound to a bead by a cleavable linker; wherein the first and second oligonucleotide form an oligonucleotide pair complementary to a nucleic acid of interest and the enzyme is capable of extending nucleic acid strands; and each cleavable linker is independently selected from a photocleavable linker and a thermally cleavable linker. Also described is a method of performing PCR and a PCR system.

### BACKGROUND

The Polymerase Chain Reaction (PCR) is used to amplify specific nucleic acid sequences and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

PCR subjects a sample to amplification conditions in the presence of an enzyme capable of elongating nucleic acid strands, for example a polymerase. The three basic steps of a single round or cycle of PCR amplification are denaturation, annealing and chain extension, each optimally taking place at different temperatures (typically 94-98° C. for denaturation; 50-65° C. for annealing, and 70-80° C. for chain extension, depending on polymerase), with each set of three steps being known by the term “thermocycling”. The amplification products (amplicons) are detected optically, typically using fluorescent reporters.

### BRIEF DESCRIPTION OF THE DRAWINGS

FIG. 1 is a schematic of an example PCR kit-of-parts;

FIG. 2 shows a mechanism of cleavage of a photocleavable linker;

FIG. 3 is a schematic of an example PCR kit-of-parts;

FIG. 4 shows an example microfluidic device for use in an example PCR system; and

FIG. 5 is a schematic of an example PCR method.

### DETAILED DESCRIPTION

Before particular embodiments of the present method and other aspects are disclosed and described, it is to be understood that the present method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:

The singular forms “a”, “an” and “the” include plural referents unless the context clearly dictates otherwise. Thus, for example, reference to “a sensor” includes reference to one or more of such sensors.

The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10wt. %, 10 wt. % to 20 wt. %, etc.

In the following description, for purposes of explanation, numerous specific details are set forth in order to provide a thorough understanding of the present apparatus and methods. It will be apparent, however, to one skilled in the art, that the present apparatus and methods maybe practiced without these specific details. Reference in the specification to “one example” or “an example” means that a particular feature, structure, or characteristic described in connection with the example is included in at least one example. The appearance of the phrase “in one example” in various places in the specification are not necessarily all referring to the same example.

Unless otherwise stated, any feature described herein can be combined with any aspect or any other feature described herein.

As used herein, the term “photocleavable linker” refers to a reactive moiety which covalently couples or binds a molecule of interest to a solid-phase bead, and which undergoes photolysis under controlled conditions to cleave or release the molecule of interest from the bead.

As used herein, the term “thermally cleavable linker” refers to a moiety which non-covalently couples or binds a molecule of interest to a solid-phase bead at room temperature, and which dissociates from the molecule of interest at elevated temperatures to cleave or release the molecule of interest from the bead.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs, for example non-natural nucleotides incorporating labels or reporter molecules, or reactive moieties may also be used (typically in the form of nucleobase modifications such as C7-modified deaza-guanine or C7-modified deaza-adenine or C5-modified cytosine or C5-modified thymidine). Non-natural nucleotides having for example sugar modifications (such as 2′-F or 2′-OMe modifications, or “LNA”, having an O—CH₂ linker between the 2′ and 4′ positions on the sugar), or backbone modifications such as phosphorothioates, or phosphorothiolates may also be used, as may unnatural bases such as the pyrimidine analog 6-amino-5-nitro-3-(1′-β-D-2′-deoxyribofuranosyl)-2(1H)-pyridone (dZ) and its Watson-Crick complement, the purine analog 2-amino-8-(1′-β-D-2′-deoxyribofuranosyl)-imidazo[1,2-a]-1,3,5-triazin-4(8H)-one (dP).

As used herein, the term “primer” refers to a short single stranded nucleic acid, typically an oligodeoxynucleotide (also referred to as an oligonucleotide herein), of about 15 to 30 nucleotides in length. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, typically of at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-CoV-2, or may encode a mutant form of the epidermal growth factor receptor (EGFR) the presence or absence of which renders a patient more or less likely to respond well to cancer treatments such as erlotinib or gefitinib.

Regardless of end application, PCR subjects a sample to multiple rounds of thermocycling in the presence of an enzyme capable of elongating nucleic acid strands, for example a polymerase. Polymerases catalyse the reaction between a deoxynucleotide triphosphate and a DNA strand, producing an elongated DNA strand bearing one more nucleotide (from the deoxynucleotide triphosphate), and pyrophosphate as a by-product. Examples of polymerases used in PCR are thermostable polymerases such as Taq polymerase (from Thermus aquaticus), Pfu polymerase (from Pyrococcus furiosus), and Bst polymerase (from Bacillus stearothermophilus). The DNA strand that is elongated in PCR is usually in the form of an oligonucleotide primer specific to a target nucleic acid sequence of interest, which is elongated using a mixture of deoxyribonucleotide triphosphates (dNTPs). For full synthesis of a standard DNA strand, four dNTPs corresponding to the four nucleobases found in DNA (adenine, guanosine, thymine and cytosine) are required: 2′-deoxyadenosine 5′-triphosphate, 2′-deoxyguanosine 5′-triphosphate, 2′-deoxycytosine 5′-triphosphate and thymidine 5′-triphosphate.

The three basic steps of a single round or cycle of PCR amplification are denaturation, annealing and chain extension, each optimally taking place at different temperatures (typically 94-98° C. for denaturation; 50-65° C. for annealing, and 70-80° C. for chain extension, depending on polymerase), hence the term thermocycling. The denaturation step separates the two strands of double-stranded DNA, with each strand acting as a template in the later chain extension step in which a complete complementary strand to the template is produced.

An oligonucleotide primer (typically comprising 15 to 30 nucleotides to ensure a balance of good specificity and efficient hybridization) is annealed to the 3′-end of each single stranded DNA molecule, and acts as a template for the synthesis of the new strand. A DNA polymerase, and a mix of dNTPs then synthesize the new strand in the chain extension step, using the original single strand of DNA as its template. Since both strands of the original DNA duplex are used as templates, a singe round or cycle of PCR results in a doubling of the number of DNA duplexes. The number of copies thus increases exponentially with the number of cycles of amplification: after 2 cycles, four DNA duplexes are present in the sample, while after 3 cycles, 8 duplexes are present.

PCR is typically done on a prepared sample of 10-50 μL and quantified by monitoring the fluorescence of the fluid as it is thermally cycled. Since the fluorescence is proportional to the amount of nucleic acid (double stranded DNA), the fluorescence intensity increases as the number of cycles of amplification (the amount of double stranded DNA produced) increases. However, in order to achieve a high enough signal to noise ratio, typically 40 cycles are required.

The amplification products (amplicons) are detected optically, typically using fluorescent reporters. Fluorescent reporter molecules used in PCR include non-specific fluorescent dyes, such as SYBR Green I, which has a distinct emission spectrum when intercalated into any double-stranded DNA, leading to an increase in fluorescence as more double-stranded DNA is produced. Other suitable reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence.

Reporter molecules may also be linked to a primer to be used in the PCR amplification, such as in the Scorpion® system, a single-stranded bi-labeled fluorescent probe sequence forming a hairpin-loop conformation with a 5′ end reporter and an internal quencher directly linked to the 5′ end of a primer via a blocker (which prevents the polymerase from extending the primer). In the beginning, the polymerase extends the primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected and is directly proportional to the amount of amplified nucleic acid.

Since different reporter dyes have different, and distinct emission spectra, combinations of different reporters can be strategically used in multiplex reactions. In addition to SYBR Green I, other cyanine dyes such as Cy3, or Cy5 can be used, as well as rhodamine dyes. Cy3 has a fluorescence emission at 570 nm, while Cy5 has a fluorescence emission at 670 nm. Other reporter dyes include the Alexa Fluor series of dyes, which have emission wavelengths ranging from 440 nm to 805 nm.

Multiplex PCR is a technique used for amplification of multiple, different, nucleic acid sequences of interest in a single experiment. For example, multiplex PCR may be used to screen for the presence of nucleic acid sequences of interest from multiple, different pathogens in a single reaction, such as simultaneously screening a single sample for the presence of viral nucleic acid sequences from any of SARS-CoV, MERS, SARS-CoV-2, influenza, and Ebola viruses.

In a multiplex PCR, many different primer pairs are required, with each pair specific to a nucleic acid sequence of interest. For example, if a sample of nucleic acid was being investigated for the presence of 10 different specific nucleic acid sequences of interest (for example 10 different viruses, or 10 different genetic mutations in a patient), then at least 10 different primer pairs would be required for the multiplex PCR.

Beads, such as microbeads (typically less than 10 μm in diameter) are commercially available and are used to conveniently capture, isolate and manipulate nucleic acids in a microfluidic environment. Microbeads are typically formed from glass, polystyrene or other inert polymer, and contain surface modifications such as thiol groups, amine groups or carboxylate groups for covalent attachment of a capture moiety. Microbeads can be magnetic, thus enabling transport of the microbead and associated nucleic acid material via use of one or more magnets.

Examples of magnetic microbeads include the Dynabead® range of products. By covalently attaching an oligonucleotide primer to a bead, a nucleic acid of interest can be pre-hybridised to the primer and thereby bound to the bead, enabling a simple sample purification before the bead is transported through a PCR system using magnets (in the case of a magnetic bead), gravity or centrifugation. Different beads can be functionalized with different primers, thus facilitating a multiplex PCR.

While this use of beads enables efficient sample purification, and localization within a PCR thermocycling chamber, the beads can cause problems during a PCR amplification process, as the beads may aggregate together, reducing access to the primer and target nucleic acid bound to the surface of the beads.

Furthermore, and independent of any bead aggregation, reactions performed on a solid surface are typically much slower than the corresponding reaction performed in the solution phase, leading to a slower and less efficient process.

The present inventors have sought to develop a PCR system that enables convenient sample manipulation alongside efficient and rapid thermocycling to provide sensitive and rapid readouts. The present inventors have found that it is possible to use orthogonally cleavable linkers to attach PCR reagents to solid phase beads for convenient sample manipulation, with cleavage of the linkers under controlled conditions allowing solution reactions to take place.

Specifically, the present inventors have found that it is possible to use photocleavable and thermocleavable linkers to attach PCR primers and enzymes to beads, to confine the beads to predetermined locations before using light and heat to cleave the linkers, releasing the primers and enzyme into solution at those predetermined locations in a thermocycling chamber. By having the PCR reactants and reagents in solution, faster hybridization and elongation can take place.

In addition, only those predetermined locations where the reagents are present need to be heated during thermocycling, resulting in even faster heating and cooling rates. This combination of rapid thermocycling with solution-phase amplification results in real-time quantitative RT-PCR in a quick and sensitive process.

In one example there is provided a PCR kit-of-parts, comprising:
a first oligonucleotide bound to a bead by a cleavable linker;
a second oligonucleotide bound to a bead by a cleavable linker; and
an enzyme bound to a bead by a cleavable linker;
   wherein
the first and second oligonucleotide form an oligonucleotide pair complementary to a nucleic acid of interest and the enzyme is capable of extending nucleic acid strands; and
each cleavable linker is independently selected from a photocleavable linker and a thermally cleavable linker.

In a further example there is provided a method of performing PCR, comprising:
introducing into a thermocycling chamber of a PCR system:
   a sample suspected of containing a nucleic acid of interest;
   an oligonucleotide pair complementary to the nucleic acid of interest; and
   an enzyme capable of extending nucleic acid strands,
wherein each oligonucleotide of the oligonucleotide pair and the enzyme are independently bound by a photocleavable linker or a thermally cleavable linker to a bead dispersed in a liquid medium in the thermocycling chamber;
irradiating the thermocycling chamber with light of a wavelength sufficient to cleave the photocleavable linker and/or providing heat to the thermocycling chamber sufficient to cleave the thermally cleavable linker to release the oligonucleotide pair and the enzyme into the liquid medium;
subjecting the liquid medium to conditions suitable for amplification by polymerase chain reaction; and
   detecting an optical signal from the liquid medium.

In a further example there is provided a PCR system comprising:
a thermocycling chamber comprising a heater;
a light source configured to emit light of a wavelength less than 400 nm to the thermocycling chamber;
a light source configured to emit light of a wavelength greater than 450 nm to the thermocycling chamber; and
an optical sensor configured to obtain optical signals from the thermocycling chamber.

### PCR Kit-of-Parts

Described herein is a PCR kit-of-parts, comprising:
a first oligonucleotide bound to a bead by a cleavable linker;
a second oligonucleotide bound to a bead by a cleavable linker; and
an enzyme bound to a bead by a cleavable linker;
   wherein
the first and second oligonucleotide form an oligonucleotide pair complementary to a nucleic acid of interest and the enzyme is capable of extending nucleic acid strands; and
each cleavable linker is independently selected from a photocleavable linker and a thermally cleavable linker.

The first oligonucleotide, the second oligonucleotide and the enzyme may be bound to the same bead. The first oligonucleotide and the enzyme may be bound to the same bead and the second oligonucleotide may be bound to a different bead. The first oligonucleotide and the second oligonucleotide may be bound to the same bead (e.g. a first bead) and the enzyme may be bound to a different bead (e.g. a second bead). For ease of handling, the first oligonucleotide may be bound to a first bead, the second oligonucleotide may be bound to a second bead, and the enzyme may be bound to a third bead.

FIG. 1 illustrates a kit-of-parts according to the present disclosure, with each oligonucleotide and the enzyme being bound to different beads. As can be seen in FIG. 1, the kit 100 comprises three beads, 102a, 102b and 102c. Bound to each bead is a linker (104a, 104b and 104c respectively), which are independently photocleavable linkers or thermally cleavable linkers. Linker 104a binds a first oligonucleotide 106 to bead 102a, while linker 104b binds a second oligonucleotide 108 to bead 104a, with oligonucleotides 106 and 108 forming an oligonucleotide pair complementary to a nucleic acid of interest, thus making them suitable as primers in a PCR amplification of the nucleic acid of interest. Finally, enzyme 110, which is capable of extending nucleic acid strands, for example a polymerase, is bound to bead 102c by linker 104c.

While FIG. 1 illustrates oligonucleotides 106 and 108, and enzyme 110 on different beads, it will be appreciated that a single bead could be functionalized with all of oligonucleotides 106 and 108, and enzyme 110.

The bead or beads may be monodisperse microbeads, such as Thermo Scientific's Opti-Link™ Carboxylate-Modified Particles, and Dynabeads®. Regardless of whether all of the first and second oligonucleotide and the enzyme are bound to the same bead or different beads, each bead may be provided with a surface modification or functionalization to allow covalent or non-covalent coupling of the first and second oligonucleotide and the enzyme to the bead.

Typical surface modifications that lend themselves to covalent coupling include carboxylate modifications, amine modifications, and pre-activated surface modifications such as a tosylate modification, with the tosylate group functioning as a leaving group to an incoming nucleophile.

Carboxylate functionalities can be converted to corresponding esters and amides using activated ester chemistry techniques, in which a carboxylate is activated using an activating compound such as the carbodiimide N,N′-dicyclehexylcarbodiimide (DCC) with DMAP, or the related carbodiimide compound 1-ethyl-3-(3-dimethylaminopropyl)carbodiimide (EDC, EDAC or EDCI), which is then reacted with a primary amine to form the corresponding amide, or an alcohol to form the corresponding ester.

An amine surface chemistry on a bead can be converted to an amide using the same chemistry, but with the carboxylate being on the group to be coupled to the bead. Surface thiol modifications can also be used, enabling coupling to, for example, a thiol-containing oligonucleotide via disulfide bond formation, while a chloromethyl functionality on the surface of a bead can react with a nucleophile, for example with a hydroxyl anion formed by deprotonation by a base of hydroxyl group).

Regardless of the nature of the surface modification on a bead, and the coupling chemistry employed, the first and second oligonucleotide and the enzyme are coupled or bound to the bead using a cleavable linker selected from a photocleavable linker and a thermocleavable linker.

Photocleavable linkers or spacers can be used to covalently couple a molecule to a solid surface or other molecule, while allowing for selective or specific cleavage at a later stage using a wavelength of light which is selective or specific for the photocleavable moiety.

Examples of photocleavable linkers include o-nitrobenzyl linkers (in particular o-nitrobenzyloxy and o-nitrobenzylamino linkers); α-substituted o-nitrobenzyl linkers, o-nitroveratryl linkers, phenacyl linkers, p-alkoxyphenacyl linkers, benzoin linkers and pivaloyl linkers.

Thus, the photocleavable linker may comprise a moiety selected from an o-nitrobenzyloxy moiety; an o-nitrobenzylamino moiety; an α-substituted o-nitrobenzyl moiety, an o-nitroveratryl moiety, a phenacyl moiety, a p-alkoxyphenacyl moiety, a benzoin moiety and a pivaloyl moiety

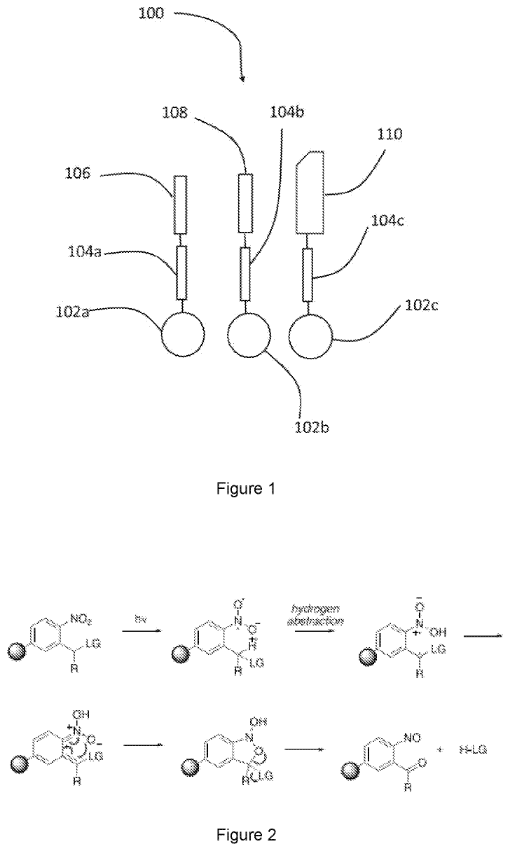

The use of a photocleavable linker ensures that the harsh cleavage conditions that would be required to cleave chemically cleavable linkers (acid/base chemistry or strong nucleophiles) that could cause DNA or RNA degradation can be avoided. Photocleavable linkers can be cleaved with specific wavelengths of light, with a cleavage mechanism for an o-nitrobenzyl linker shown in FIG. 2.

Structures of example photocleavable linkers and their cleavage conditions are shown in Table 1, with R in all cases being the moiety of interest, for example the oligonucleotides or the enzyme, and the sphere representing the bead.

TABLE 1

| | | Cleavage |
|---|---|---|
| Linker | Structure | wavelength |
| o-nitrobenzyl | 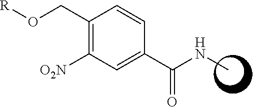 | 350 nm |
| o-nitroveratryl | 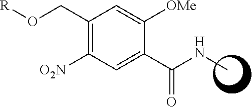 | 350 nm |
| Phenacyl | 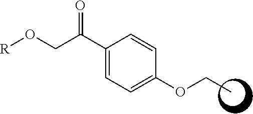 | 300-350 nm |
| Benzoin | 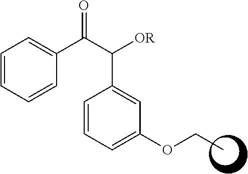 | 350 nm |
| Pivaloyl | 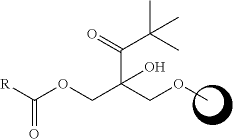 | 280-340 nm |

While the photocleavable moiety is shown in Table 1 in close proximity to the bead, it will be appreciated that a spacer, such as an alkyl spacer (for example a C₆-C₁₀ alkyl spacer), or a PEG spacer may be incorporated to distance the photocleavable linker and coupled oligonucleotide/enzyme from the surface of the bead. Methods of coupling such photocleavable linkers to activated beads include chemistries such as those mentioned above.

In the instance that a photocleavable linker is used to couple an oligonucleotide to the bead, a straightforward approach to achieving this would be via the free primary hydroxyl group at the 5′-end of the oligonucleotide, with this having a greater reactivity than the secondary hydroxyl group at the 3′-end. Methods of coupling primary hydroxyl groups, in particular 5′-hydroxyl groups to linkers are known in the art, and typically proceed via deprotonation of the hydroxyl group using a base, with the generated oxygen anion able to act as a nucleophile to displace a leaving group on the linker. Alternatively, coupling of an oligonucleotide to a solid support can be achieved via the secondary hydroxyl group of the 3′-end, by first protecting the 5′-hydroxyl group and then coupling via the 3′-hydroxyl group, as is done for solid phase synthesis of oligonucleotides.

One or both of the first oligonucleotide and the second oligonucleotide may be bound to a bead by a photocleavable linker, for example an o-nitrobenzyl linker. In one example, the first oligonucleotide is bound to a first bead by a first photocleavable linker, and the second oligonucleotide is bound to a second bead by a second photocleavable linker, with the first photocleavable linker and the second photocleavable linker being cleaved under different or orthogonal cleavage conditions such as different wavelengths of light.

In the instance that the enzyme is bound to a bead by a photocleavable linker, the site of binding may conveniently be the N-terminus or the C-terminus of the enzyme, or an internal lysine residue, for example. As described above, amines and acids (such as at the N-and C-termini of a polypeptide) can be activated to form amide bonds. As the enzyme will be cleaved from the bead during photolysis, it will retain or regain its active conformation after cleavage. Suitable enzymes include the thermostable polymerases Taq, Bst and Pfu.

In some examples, any of the first oligonucleotide, the second oligonucleotide and the enzyme may be bound to a bead by a thermally cleavable linker. In some examples, the thermally cleavable linker may be an antibody, an affibody or an aptamer, specific to the moiety being bound (i.e. an oligonucleotide or the enzyme).

Affibodies are small proteins that have been engineered to bind to a target, in the same way as an antibody, and are known an antibody mimetics. Affibodies are typically much smaller than antibodies, typically comprising only a few alpha helices, but are still capable of folding into a three-dimensional structure that can be engineered to specifically bind to a target.

Aptamers are single stranded nucleic acids (DNA or RNA) that fold into a three-dimensional shape that can specifically bind to a target molecule analogously to antibodies, that is by recognizing and binding the three-dimensional, tertiary structure of the target rather than a complementarity binding based on Watson-Crick or Hoogsteen base-pairing. Methods of generating antibodies and aptamers to protein targets and nucleic acid targets are known, and include hybridoma technology (for antibody generation) and SELEX procedures (“Systematic Evolution of Ligands by EXponential enrichment” for aptamer generation). Antibodies, affibodies and aptamers are able to function as thermally cleavable linkers in that there is no covalent bond formed between the target molecule and the antibody, affibody or aptamer, with association/binding relying on non-covalent van der Waal's forces which can be easily overcome with heating to denature or dissociate the binding.

An example of a thermally cleavable linker that may be utilized in the kit and methods of the present disclosure includes a binding agent specific to an enzyme, more particularly a DNA polymerase such as Taq DNA polymerase, as is used in Hot Start DNA processes. Hot Start technology utilizes binding agents such as antibodies or affibodies to bind to the active site of a polymerase at room temperature, thereby blocking enzyme activity. Since the binding is non-covalent, an increase in temperature, such as the initial denaturing step of a PCR reaction, is sufficient to break the non-covalent bonds between enzyme and binding agent and restore enzyme activity.

Typically, the enzyme can be released and its activity restored at temperatures of about 60° C. or above for as little as one minute. Examples of antibody-based Hot Start processes include the DreamTaq Hot Start DNA polymerase, the Platinum II Taq DNA polymerase and the Platinum SuperFi II DNA polymerase. Examples of affibody-based Hot Start processes include the Phire Hot Start II DNA polymerase, and the Phusion Hot Start II DNA polymerase. Using a thermally cleavable linker can reduce amplification products from primer dimerization and other non-specific amplification in the same way as non-bead based Hot Start processes.

It will be understood that antibodies, affibodies and aptamers can be coupled to a solid support (e.g. a bead) using approaches described above, with the option of including an inert chain spacer, such as an alkyl spacer (for example a C₆-C₁₀ alkyl spacer), or a PEG spacer, to ensure that the bead does not interfere with the conformational folding of the antibody, affibody or aptamer.

In some examples, the kit-of-parts comprises a plurality of first and second oligonucleotides forming a plurality of oligonucleotide pairs, with each oligonucleotide pair being complementary to a different nucleic acid of interest with each oligonucleotide of each oligonucleotide pair being bound to a bead by a cleavable linker. Thus, each oligonucleotide pair can be considered as a primer pair for PCR amplification of a nucleic acid of interest.

Each oligonucleotide pair may be bound to a bead by the same type of cleavable linker, for example a nitrobenzyl linker, with different oligonucleotide pairs being bound to their respective beads by different cleavable linkers. For example, each oligonucleotide of a first oligonucleotide pair may be bound to a bead (whether the same bead or different beads) using a nitrobenzyl linker, while each oligonucleotide of a second oligonucleotide pair may be bound to a bead (whether the same bead or different beads) using a pivaloyl linker.

In this way, temporal multiplexing may be achieved. For example, the first pair of oligonucleotide primers is first released and an amplification of the target nucleic acid of interest attempted. If the target did not amplify, the second pair of oligonucleotides may be released and another amplification attempted; and so on until the amplification is successful. A high selectivity between the primer sets is achieved due to the fact that the bulk reaction (in solution) is typically orders of magnitude more efficient than surface reactions.

As these linkers can be selectively cleaved using different wavelengths of light, as set out in Table 1, it becomes possible to selectively cleave only one oligonucleotide pair at a time, to determine whether or not the nucleic acid of interest corresponding to that oligonucleotide pair is present in a sample. The selectivity between the primer sets is achieved due to the fact that the bulk reaction is typically orders of magnitude more efficient than surface reactions.

The above described beads, functionalised with oligonucleotides serving as PCR primers, and a DNA elongation enzyme such as a polymerase provide convenient means of delivering these PCR reagents to a thermocycling chamber. Accordingly, these beads can be considered as reagent delivery beads or reagent beads. In some examples, the kit-of-parts further includes a capture bead, to capture a nucleic acid of interest from a sample during purification of a sample prior to amplification. While in some examples an oligonucleotide of an oligonucleotide pair serving as PCR primers may serve to capture the nucleic acid of interest, other capturing moieties that could be used include chitosan-modified beads or silica particles.

By using different types of beads, for example, capture beads and reagent delivery beads manufacturing and flexibility of the kit-of-parts and system can be improved, as purification of a target nucleic acid can be performed before any reagent beads are mixed with the target nucleic acid.

Capture beads comprising silica, chitosan, alumina and other type of surface chemistry can accumulate target DNA during a sample preparation phase and mixed with reagent delivery beads in a thermocycling chamber of a PCR system. The system undergoes the thermal and/or photo activation processes, whereby the target DNA is released from the surface of the capture beads and the oligonucleotides and polymerase are released from the surface of the reagent delivery beads, enabling PCR reaction in the vicinity of the beads as is described below in connection with the method of the present disclosure.

In some examples, the kit-of-parts may further comprise a reverse transcriptase bound to a bead by a photocleavable or thermally cleavable linker. A kit-of-parts including a bead-bound reverse transcriptase enables the reverse transcription of an RNA sample of interest into cDNA prior to PCR amplification. In this example, the reverse transcriptase and oligonucleotide pairs can be bound to beads by photocleavable linkers, while a DNA polymerase is bound to a bead by a photocleavable linker. The kit-of-parts may comprise a capture bead functionalised with a poly dT oligonucleotide, to capture an RNA molecule having a poly A tail.

The kit-of-parts may also include a reporter molecule, such as a fluorescent dye as described herein. Thus, the kit-of-parts may comprise a non-specific fluorescent dye, such as SYBR Green I, or a target-specific fluorescent reporter molecule, such as a TaqMan hydrolysis probe.

In some examples, the reporter molecule may be linked to an oligonucleotide of the oligonucleotide pair, such as in the Scorpion® system. In some examples, the reporter molecule is a target-specific fluorescent reporter molecule comprising an oligonucleotide complementary to a target region of the nucleic acid of interest, with the oligonucleotide of the reporter molecule also being bound to a bead such as described above.

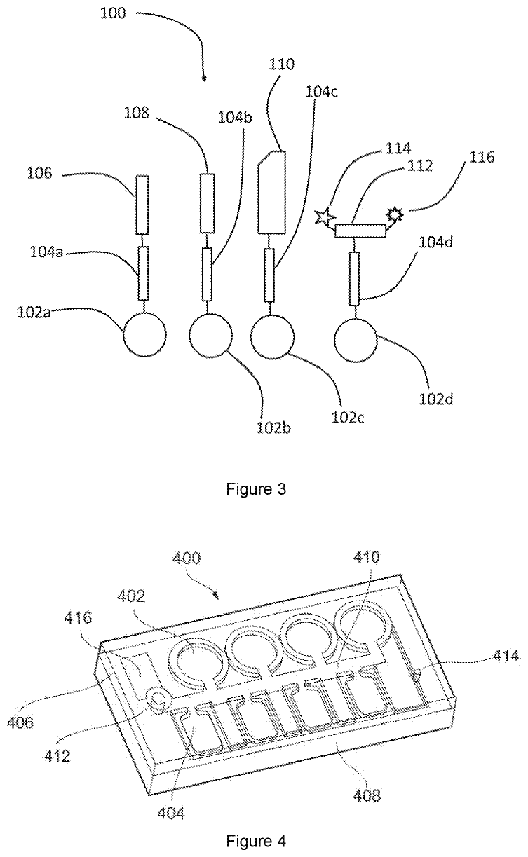

FIG. 3 illustrates this alternative kit-of-parts (also denoted generally at 100), in which a fourth bead 102d is included. Bead 102d is attached to a fourth linker 104d (again, a photocleavable or a thermally cleavable linker), which binds a reporter probe 112 to bead 104d. Probe 112 comprises a target-specific fluorescent reporter molecule comprising an oligonucleotide complementary to a target region of the nucleic acid of interest, which is functionalised with the fluorophore 114 and a quencher 116.

The kit-of-parts may further comprise one or more of dNTPs, MgCl₂ as an enzyme co-factor (although other co-factors, such as MgSO₄ may be used with certain enzymes), all dissolved in an aqueous buffer.

### PCR System

Described herein is a PCR system comprising:
a thermocycling chamber comprising a heater;
a light source configured to emit light of a wavelength less than 400 nm to the thermocycling chamber;
a light source configured to emit light of a wavelength greater than 450 nm to the thermocycling chamber; and
an optical sensor configured to obtain optical signals from the thermocycling chamber.

Also described herein is a PCR system, comprising:
a thermocycling chamber comprising a heating element;
a light source configured to emit light of a wavelength sufficient to cleave chemical bonds;
a light source configured to emit light of a wavelength suitable to excite a fluorophore; and
an optical sensor configured to obtain optical signals from the thermocycling chamber.

The PCR system may comprise a single chamber or a plurality of chambers provided on a substrate. A plurality of thermocycling chambers may be independently operable. In other words, each thermocycling chamber of a plurality of thermocycling chambers may have its own dedicated heater or heaters, so as to be independently controllable. In this way, synchronous or asynchronous control of a plurality of different PCR assays can be performed. Asynchronous control of individual thermocycling chambers within a plurality of thermocycling chambers enables the amplification of a plurality of different samples using different thermocycling, or even isothermal, protocols. In some examples, each thermocycling chamber is provided with a plurality of independently operable heaters or heating elements, thus enabling a plurality of different PCR assays to be performed in a single thermocycling chamber, as described in the methods of the present disclosure.

In some examples, the thermocycling chamber is present as part of a microfluidic network, in which one or more microfluidic channels and chambers are fluidically connected to the thermocycling chamber, and through which a sample for purification, amplification and analysis is transported to the thermocycling chamber. Thus, in some examples, the thermocycling chamber may be provided in or as part of a microfluidic device. In some examples, the PCR system, for example a microfluidic device of the PCR system, comprises a plurality of thermocycling chambers, each fluidly connected to a central flow channel via which reagents and/or samples for amplification may be provided. In some examples, the PCR system, for example a microfluidic device of the PCR system, comprises a plurality of thermocycling chambers, each fluidly connected to its own dedicated flow channel via which reagents and/or samples for amplification may be provided. In other words, the thermocycling chamber is fluidically isolated from any other thermocycling chamber that may be present.

The PCR system, for example a microfluidic device of the PCR system may comprise a sample purification chamber in fluid connection with the thermocycling chamber. The sample purification chamber provides a location within the device to purify and isolate a nucleic acid from, for example, a cell lysis protocol prior to transporting the nucleic acid to the thermocycling chamber using a bead to which the nucleic acid can reversibly bind. In the instance in which a plurality of thermocycling chambers are present in the PCR system, which may be or may comprise a microfluidic device or cassette, each thermocycling chamber may be provided with a dedicated sample purification chamber upstream from the thermocycling chamber. Alternatively, a single sample purification chamber may serve a plurality of thermocycling chambers.

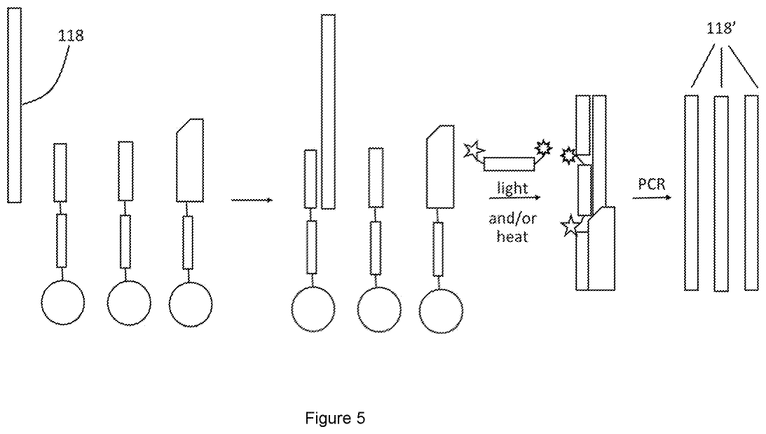

FIG. 4 shows an example of a microfluidic device 400 of a PCR system, incorporating a plurality of thermocycling chambers 402. In the example device, four thermocycling chambers 402 are approximately circular in shape (top row), with the device being provided with four elongate sample purification chambers 404 (bottom row), all provided in a cover layer 406 on a substrate 408, though it will be appreciated that other arrangements of chambers are possible. In the device shown, each thermocycling chamber 402 and each sample purification chamber 402 is fluidically connected to a central flow channel 410, served by an inlet port 412 in cover layer 406, though it will be appreciated that this is only one example and that other configurations are possible. In the device of FIG. 4, each thermocycling chamber 402 is also fluidically connected to a common vent 414, also in cover layer 410, though it will be appreciated that each chamber may have its own dedicated vent.

As just described in connection with FIG. 4, a PCR system may comprise a substrate 408 on which the thermocycling chamber 402 is provided or the plurality of thermocycling chambers are provided. In some examples, the PCR system comprises a substrate on which the heater and thermocycling chamber are provided. Some example substrates may include polypropylene substrates, polycarbonate substrates, polydimethylsiloxane (PDMS) substrates, silicon-based substrates, glass-based substrates, gallium arsenide based substrates, cyclic-olefin copolymer substrates (COC substrates) and/or other such suitable types of substrates for microfabricated devices and structures. In some examples, the substrate may be formed from a photoresist material such as SU8, an epoxy-based photoresist material, in which a pattern has been etched and into which at least a portion of the electrode assembly is to be incorporated. In some examples, the substrate is formed from a transparent material, thereby permitting optical detection from underneath the thermocycling chamber.

Thermocycling chamber 402 may be defined by or provided in cover layer 406 disposed on a substrate 408. Cover layer 406 may also be termed a fluidic layer or fluidic stack, and may be formed by selectively etching or machining away regions of material so as to form a reaction chamber. Cover layer 406 may comprise any material or combination of materials suitable for use in microfluidic devices, including polycarbonate, and cyclic olefin copolymer.

As used herein, the terms “cover layer”, “microfluidic layer”, “microfluidic stack”, “fluidic layer” or “fluidic stack” refer to the components of the microfluidic device through which one or more fluids can pass during use of the microfluidic device, for example through one or more microfluidic channels and chambers. The terms are intended to encompass multiple flow paths, for example in different levels of the layer/stack, and distinguish these flow channel-containing components from other operational modules such as electronic circuitry and sensors.

Other layers present in a fluidic stack may include layers of adhesive (for example pressure-sensitive adhesives) to bond the fluidic layer to the substrate and/or bond layers of a fluid stack to each other. Suitable adhesives include pressure-sensitive adhesives, which typically comprise an elastomer based on acrylic, silicone or rubber optionally compounded with a tackifier such as a rosin ester. Convenient pressure sensitive adhesives are in the form of double-sided films or tape, such as the acrylic adhesives 200MP and 7956MP available from 3M™.

In some examples, the cover layer is provided with one or more fluid inlets and outlets, such as inlet port 412 and common vent 414. The presence of a vent enables unhindered flow of liquid through the chambers and minimises risk of unwanted bubble formation within the chambers, particularly thermocycling chamber 302.

In some examples, the heater or heater element is provided on or within a substrate, to provide heat to the thermocycling chamber. The heater may be provided on a surface of the substrate. In some examples, the heater comprises a flat panel heater or one or more thermally conductive printed electrical traces or wires. The substrate may comprise or is a printed circuit board (PCB), and so in some examples is termed a PCB substrate, and the heater comprises one or more printed electrical traces on a substrate to provide heat to the thermocycling chamber. In some examples, the heater is provided above or below the plane of the microfluidic device, or embedded into a substrate on which the thermocycling chamber is disposed. The heater may comprise a Peltier device, a flat panel heater in the form of a solid-state active heat pump. The heater may comprise at least one micro-heater in thermal contact with a predetermined location of the thermocycling chamber. The at least one-micro-heater may comprise one or more electrical wire on a surface of the thermocycling chamber. In some examples, the heater receives electrical power from electrically conductive wires provided on or to the substrate to form an electrical circuit which supplies electrical current to the heater. Such components may be controlled by a controller located on or off the substrate via control signals.

The flat panel heater or the electrical traces or wires may be formed from any suitable material used for producing microelectronics for microfluidic applications. For example, the heater (whether a flat panel heater, printed electrical trace or wire) may be formed from a metal such as platinum, aluminium, titanium, chromium, nickel, copper, silver or gold or a combination thereof. The heater may be formed by sputtering or inkjet printing directly onto a substrate, or onto an adhesive layer covering the substrate. Stainless steel and carbon electrodes may also be used, for example as laser cut or die punched stainless steel foils. In one example, the heater comprises a titanium adhesion layer onto which a conductive gold film is deposited. A flat panel heater or printed electrical trace may have a thickness in the region of 500 nm or less, for example 400 nm or less, for example 300 nm or less, for example 200 nm or less, for example about 100 nm.

In one example, the heater is in direct thermal contact with a liquid medium to be heated in the thermocycling chamber. The thickness of the heater then correlates with √(D·t), where D is the thermal diffusivity of the liquid medium containing the beads and unbound primers, enzyme and nucleic acid of interest (in the order of 1.5×10⁻⁷) m²/s) and t is the heating duration. In other examples, particularly when the material used to form the heater is a reactive metal, such as copper, a passivation layer or dielectric layer can be deposited onto the heater. The dielectric layer may be spun on or sputtered onto the heater and substrate. Thus, in some examples, the heater may be provided with a dielectric or passivation coating of polyimide, SU-8, silicon oxide, silicon nitride, aluminium oxide, aluminium nitride or any combination/stack thereof. Another suitable material is Kapton®, which may be incorporated into a coating or stack with any of the aforementioned materials.

In some examples, each thermocycling chamber may have a plurality of independently operable heaters, with each heater provided at a predetermined location. In other words, each thermocycling chamber can have multiple reaction zones which are independently or asynchronously controllable relative to the each other. In this way, a plurality of different PCR assays (i.e. a multiplexed PCR), each requiring a different thermocycling protocol, can be performed in a single reaction chamber. For example, different thermocycling protocols may require shorter or longer annealing times, or higher or lower annealing temperatures, based on length and content of the primers used (longer oligonucleotides, or oligonucleotides having high proportions of G:C base pairs will have higher melting temperatures, which will affect annealing of the primer to the template strand).

In some examples, the predetermined locations at which heaters are provided are spaced apart to avoid cross-contamination. In some examples, the individual locations are spaced from 100 μm to 1000 μm apart, in some examples from 200 to 800 μm apart, in some examples 300 to 600 μm apart, and in some examples 500 μm apart. Since the methods of the present disclosure may utilise amplification conditions which do not include fluid flow within the thermocycling chamber, spacing apart the individual heaters in this way avoids cross-contamination as amplification of nucleic acid material sufficient to obtain a positive or negative detection result is quicker than any diffusion of the nucleic acid of interest, and quicker than diffusion of oligonucleotide primers used to amplify the nucleic acid of interest.

In some examples, the PCR system may be provided with a magnet or a plurality of magnets in or under the substrate. The magnet may comprise a permanent magnet or an electromagnet. When used in combination with one or more magnetic beads to which an oligonucleotide primer or enzyme is bound as described herein, the magnet or magnets can draw the beads to a predetermined location within the sample purification chamber or the thermocycling chamber and thus increase a local concentration of bead and bound oligonucleotide/enzyme.

The PCR system comprises a light source configured to emit light of a wavelength sufficient to cleave chemical bonds, to enable cleavage of a photocleavable linker, thereby releasing a bound oligonucleotide, enzyme or reporter molecule into solution in a liquid medium in the thermocycling chamber. Typically, light in the UV region of the electromagnetic spectrum is used to cleave photocleavable linkers. Thus, in some examples, the PCR system comprises a light source configured to emit light of a wavelength less than 400 nm to the thermocycling chamber, for example less than 380 nm, for example less than 370 nm, for example less than 360 nm, for example from 280 nm to 360 nm. The light source configured to emit light of a wavelength less than 400 nm may comprise a mercury lamp, a laser or a UV LED.

The light source may be configured, or operable, to emit light with a power of at least 10 mW/mm², for example at least 20 mW/mm², for example at least 30 mW/mm², for example at least 40 mW/mm², for example at least 50 mW/mm², for example at least 60 mW/mm², for example at least 70 mW/mm², for example at least 80 mW/mm², for example at least 90 mW/mm², for example at least 100 mW/mm², for example at least 110 mW/mm², for example at least 120 mW/mm², for example at least 130 mW/mm², for example at least 140 mW/mm², for example at least 150 mW/mm², for example at least 160 mW/mm², for example at least 170 mW/mm², for example at least 180 mW/mm², for example at least 190 mW/mm², for example up to 200 mW/mm².

The light source may be configured, or operable, to emit light with a power of less than 200 mW/mm², for example less than 190 mW/mm², for example less than 180 mW/mm², for example less than 170 mW/mm², for example less than 160 mW/mm², for example less than 150 mW/mm², for example less than 140 mW/mm², for example less than 130 mW/mm², for example less than 120 mW/mm², for example less than 110 mW/mm², for example less than 100 mW/mm², for example less than 90 mW/mm², for example less than 80 mW/mm², for example less than 70 mW/mm², for example less than 60 mW/mm², for example less than 50 mW/mm², for example less than 40 mW/mm², for example less than 30 mW/mm², for example less than 20 mW/mm², for example about 10 mW/mm².

The light source may be configured, or operable, to emit light with a power of from 10 to 200 mW/mm², for example from 20 to 190 mW/mm², for example from 30 to 180 mW/mm², for example from 40 to 170 mW/mm², for example from 50 to 160 mW/mm², for example from 70 to 150 mW/mm², for example from 75 to 140 mW/mm² for example from 80 to 130 mW/mm², for example from 85 to 120 mW/mm², for example from 90 to 110 mW/mm², for example about 100 mW/mm².

The light source may be configured, or operable, to emit light for any of the above described power values for a period of at least 1 second, for example at least 2 seconds, for example at least 3 seconds, for example at least 4 seconds, for example at least 5 seconds, for example at least 6 seconds, for example at least 7 seconds, for example at least 8 seconds, for example at least 9 seconds, for example at least 10 seconds, for example at least 11 seconds, for example at least 12 seconds, for example at least 13 seconds, for example at least 14 seconds, for example at least 15 seconds, for example at least 16 seconds, for example at least 17 seconds, for example at least 18 seconds, for example at least 19 seconds, for example at least 20 seconds.

The light source may be configured, or operable, to emit light for any of the above described power values for a period of less than 20 seconds, for example less than 19 seconds, for example less than 18 seconds, for example less than 17 seconds, for example less than 16 seconds, for example less than 15 seconds, for example less than 14 seconds, for example less than 13 seconds, for example less than 12 seconds, for example less than 11 seconds, for example less than 10 seconds, for example less than 9 seconds, for example less than 8 seconds, for example less than 7 seconds, for example less than 6 seconds, for example less than 5 seconds, for example less than 4 seconds, for example less than 3 seconds, for example less than 2 seconds, for example about 1 second.

The light source may be configured, or operable, to emit light for any of the above described power values for a period of from 1 to 20 seconds, for example from 2 to 19 seconds, for example from 3 to 18 seconds, for example from 4 to 17 seconds, for example from 5 to 16 seconds, for example from 6 to 15 seconds, for example from 7 to 14 seconds, for example from 8 to 13 seconds, for example from 9 to 12 seconds, for example about 10 seconds,

The light source may be configured, or operable, to emit light with a power of from 10 to 200 mW/mm², for example from 20 to 190 mW/mm², for example from 30 to 180 mW/mm², for example from 40 to 170 mW/mm², for example from 50 to 160 mW/mm², for example from 70 to 150 mW/mm², for example from 75 to 140 mW/mm², for example from 80 to 130 mW/mm², for example from 85 to 120 mW/mm², for example from 90 to 110 mW/mm², for example about 100 mW/mm² for a period of from 1 to 20 seconds, for example from 2 to 19 seconds, for example from 3 to 18 seconds, for example from 4 to 17 seconds, for example from 5 to 16 seconds, for example from 6 to 15 seconds, for example from 7 to 14 seconds, for example from 8 to 13 seconds, for example from 9 to 12 seconds, for example about 10 seconds,

The light source may be configured, or operable, to emit light having an overall power density of from 1 mJ/cm² to 200 mJ/cm², for example 1 mJ/cm² to 180 mJ/cm², for example 1 mJ/cm² to 170 mJ/cm², for example 1 mJ/cm² to 160 mJ/cm², for example 10 mJ/cm² to 150 mJ/cm², for example 15 mJ/cm² to 140 mJ/cm², for example 20 mJ/cm² to 130 mJ/cm², for example 25 mJ/cm² to 140 mJ/cm², for example 50 mJ/cm² to 120 mJ/cm², for example 75 mJ/cm² to 110 mJ/cm² for example 80 mJ/cm² to 100 mJ/cm².

In some examples, the PCR system comprises a light source configured to emit light of a wavelength sufficient to cleave a photocleavable linker as described herein, for example light of a wavelength greater than 450 nm; and an optical sensor configured to obtain optical signals from the thermocycling chamber. In some examples, the optical sensor is a fluorescence sensor and the optical signals are fluorescence signals. As described above, fluorescent molecules are used as reporter molecules in PCR amplification, with the fluorescence intensity proportional to the amount of amplified nucleic acid material. Wavelengths of light suitable to cause fluorescence of reporter molecules used in PCR amplification are generally greater than 450 nm, with the wavelength of fluorescence being even greater.

In some examples, the light source comprises a laser diode, or an LED, configured to emit light of a wavelength suitable to cause fluorescence of a fluorescent reporter molecule during a PCR amplification process. For example, SYBR Green I, absorbs blue light with a λₘₐₓ of 497 nm, and emits green light with a λₘₐₓ of 520 nm. In some examples, the detector may be a charge coupled device (CCD) or pin photodiode configured to detect the emitted fluorescent light. In some examples, the optical sensor comprises a detector, which may be a charge coupled device (CCD) or pin photodiode to detect the emitted fluorescent light. In some examples, the optical sensor is arranged above or below the thermocycling chamber, for example above or below a plane in which the liquid sample is being thermocycled. In some examples, a microfluidic device on which the thermocycling chamber is provided comprises an optical window or opening that allows transmission of light therethrough to an optical sensor located in the PCR system but external to the microfluidic device, or within the microfluidic device itself. In some examples, the optical sensor is embedded into a cover of the microfluidic device.

The thermocycling chamber may be provided on or in a cassette, or chip, to be used in the PCR system. In some examples, the microfluidic device may be a single use or disposable device. In some examples, the microfluidic device may be configured to be inserted into or received by a port in the system. In some examples, the microfluidic device may be provided with one or more fluidic connections that are configured to engage with one or more corresponding fluidic connections in the apparatus, to enable fluid flow from the system into the microfluidic device, for example to enable transfer of a sample injected into an injection port of the system to be transferred to a sample purification chamber or a thermocycling chamber of the microfluidic device. In other examples, the or each chamber of the microfluidic device may be filled with sample prior to inserting the microfluidic device into the system, for example by manual pipetting a sample solution through an inlet port such as a Luer connector or membrane valve.

In examples in which the thermocycling chamber is provided on a device such as a microfluidic device, the PCR system may comprise an electrical interface, configured to contact an electrical interface provided on the device. The electrical interface on the system may be coupled to any component of the device that requires electrical current to operate. Examples of such components include the heater elements, either in flat panel form or printed conductive trace form, and actuators for controlling fluid flow within the device, or for transporting reagent or sample beads from one chamber to another. In some examples, the electrical interfaces may be multi-pin input/output off board connecters, for example 44-pin connectors that enable electrical coupling of the device to a computer module of the PCR system. Each pin of the electrical interface may provide an electrical contact to a specific component of the device, such as the flat panel heater or micro-heaters described herein. The electrical coupling of the device to the system allows control signals from the computer module to be sent to the PCR device so that electrical current can be sent to desired modules of the device.

As noted above, the PCR system may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the device and/or system such as one or more heater assemblies and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module facilitates the introduction of a sample into a sample purification chamber and a thermocycling chamber, or into multiple thermocycling chambers. For example, the computer control module may control a series of valves and pumps in the system or on the device to direct flow of a test sample or solution to the thermocycling chamber after purification.

In some examples, the computer control module may further control the processing of a sample in a thermocycling chamber, for example by subjecting the thermocycling chamber to thermocycling conditions. For example, the computer control module may control, through the output of control signals, the operation of one or more heater elements to control the temperature and duration of heating within the or each thermocycling chamber. For example, the computer control module may control, through the output of control signals the provision of one or more pulses of electrical energy to the heater to generate waves of thermal energy in a liquid medium within the thermocycling chamber, radiating from the surface adjacent the heater into the liquid medium.

The computer control module may also control, through the output of control signals, the operation of a light source to control emission of light of a wavelength less than 400 nm to the thermocycling chamber to cleave a photocleavable linker. The computer control module may also control, through the output of control signals, the operation of a light source to control emission of light of a wavelength greater than 450 nm to the thermocycling chamber to monitor progress of an amplification reaction via excitation of a fluorophore with the light of a wavelength greater than 450 nm.

In other examples, the computer control module may control, through the output of control signals, the operation of the optical sensor, to monitor progress of the PCR amplification via detection of the excited fluorophore and thereby detection of the presence of a nucleic acid of interest in a sample. As a result, a sample and reagents may undergo various selected reactions, various selected heating cycles and various sensing operations under the control of the computer control module.

### Method of Manufacturing a PCR System

A method of manufacturing a PCR system is described, comprising:
forming a thermocycling chamber provided with a heater; and
configuring:
   a light source to emit light of a wavelength less than 400 nm to the thermocycling chamber;
   a light source to emit light of a wavelength greater than 450 nm to the thermocycling chamber; and
   an optical sensor to obtain optical signals from the thermocycling chamber.

An alternative method of manufacturing a PCR system is described, comprising:
forming a thermocycling chamber provided with a heating element; and
configuring:
   a light source configured to emit light of a wavelength sufficient to cleave chemical bonds;
   a light source configured to emit light of a wavelength suitable to excite a fluorophore; and
   an optical sensor configured to obtain optical signals from the thermocycling chamber.

In some examples, the heater may be in the form of a flat panel heater disposed on a substrate as described previously. In some examples, the substrate may be etched to provide one or more recesses or traces into which the heater can be inserted or printed.

In some examples, forming a thermocycling chamber may comprise forming or providing a cover layer having one or more thermocycling chambers, and arranging the cover layer on the substrate so as to align a thermocycling chamber with at least one heater on the substrate. In some examples, the upper surface of the substrate forms the bottom surface of the thermocycling chamber. In some examples, the thermocycling chamber has an internal diameter of from 5 to 200 μm, for example from 5 to 100 μm, for example from 10 to 100 μm.

The methods comprise configuring: a light source configured to emit light of a wavelength sufficient to cleave chemical bonds; a light source configured to emit light of a wavelength suitable to excite a fluorophore. For example, the methods comprise configuring: a light source to emit light of a wavelength less than 400 nm to the thermocycling chamber, and a light source to emit light of a wavelength greater than 450 nm to the thermocycling chamber.

The light sources may be independently selected from mercury lamps, LEDs and lasers. The light sources may be directly integrated into the thermocycling chamber, for example into a wall or cover of the thermocycling chamber or they may be located elsewhere in the system but configured to emit light of the appropriate wavelengths to the thermocycling chamber. In some examples, a single light source is provided, which is configured to emit light of different wavelengths at different times. In particular, the single light source may be operable to emit light of a wavelength sufficient to cleave chemical bonds (for example light of a wavelength less than 400 nm) in order to cleave one or more photocleavable linkers to release PCR reagents into a liquid medium, prior to a second wavelength being emitted with the second wavelength being suitable to excite a fluorophore during amplification. The use of one or more bandpass filters enables operation of a single light source in this manner.

The method comprises configuring an optical sensor to obtain optical signals from the thermocycling chamber. In some examples, the optical sensor is a fluorescent sensor. In some examples, the optical sensor is directly integrated into the thermocycling chamber, for example into a wall or cover of the thermocycling chamber or is located elsewhere in the system but configured to receive signals from the thermocycling chamber.

### Method of Performing PCR

Described herein is a method of performing PCR, comprising:
introducing into a thermocycling chamber of a PCR system:
   a sample suspected of containing a nucleic acid of interest;
   an oligonucleotide pair complementary to the nucleic acid of interest; and
   an enzyme capable of extending nucleic acid strands,
wherein each oligonucleotide of the oligonucleotide pair and the enzyme are independently bound by a photocleavable linker or a thermally cleavable linker to a bead dispersed in a liquid medium in the thermocycling chamber;
irradiating the thermocycling chamber with light of a wavelength sufficient to cleave the photocleavable linker and/or providing heat to the thermocycling chamber sufficient to cleave the thermally cleavable linker to release the oligonucleotide pair and the enzyme into the liquid medium;
subjecting the liquid medium to conditions suitable for amplification by polymerase chain reaction; and
   detecting an optical signal from the liquid medium.

The PCR system may be as described herein.

FIG. 5 illustrates the basic principle of the method. In FIG. 5, all components which are common to FIGS. 1 and 3 have not been labelled, for simplicity. A nucleic acid of interest, labelled 118, is indicated with a kit of parts, comprising first and second oligonucleotide primers and an enzyme, each bound to a different bead by a photocleavable or thermally cleavable linker. While FIG. 5 shows nucleic acid 118 as being in solution, it may also be bound to a nucleic acid capture bead as described previously. In yet another example, nucleic acid 118 may be pre-mixed with the kit-of-parts to hybridise it to an oligonucleotide primer before being introduced into a thermocycling chamber. As can be seen in FIG. 5, nucleic acid 118 hybridises to an oligonucleotide primer, thus bringing it into close proximity with the PCR reagents. As noted, this may occur in a sample purification chamber or in the thermocycling chamber.

FIG. 5 schematically shows a reporter probe being added to the system. Again, this may be introduced into a thermocycling chamber as part of a kit-of-parts, bound to a bead, or it may be introduced separately to the kit-of-parts, for example dissolved in a liquid medium introduced into the thermocycling chamber. FIG. 5 also indicates that the action of light and/or heat cleaves the photocleavable/thermally cleavable linkers, to release the oligonucleotide primers and enzyme into solution. At this point, a complex of the target nucleic acid, the oligonucleotide primers, the reporter probe and the enzyme forms in solution in the liquid medium in the thermocycling chamber, and amplification by PCR can occur-in solution-leading to generation of copies 118′ of the original target nucleic acid of interest.

A sample suspected of containing a nucleic acid of interest may be introduced into the thermocycling chamber in a liquid medium. The sample may comprise a nucleic acid for analysis, e.g. a nucleic acid of interest, which is to be amplified in a method as described herein. In some examples, the sample may comprise a plurality of nucleic acids for analysis which are to be amplified in a method as described herein. In some examples, the sample is suspected of comprising one or a plurality of nucleic acid sequences of interest. In some examples, the sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. That is, the sample suspected of containing a nucleic acid of interest may be purified in a purification chamber with the purified nucleic acid of interest being introduced into the thermocycling chamber.

In some examples, the nucleic acid sample is not isolated from the bodily fluid or tissue via which it was obtained. The nucleic acid sample obtained from a subject may be incorporated into a test solution with or without any isolation or preparation. In some examples, the nucleic acid sample obtained from a subject is dissolved or dispersed in an aqueous solution, thus forming a test solution.

The liquid medium introduced into the thermocycling chamber may comprise an aqueous solution of a nucleic acid of interest. In other examples, the nucleic acid sample may be bound to a capture bead as described herein with the capture bead being dispersed or suspended in the liquid medium.

An oligonucleotide pair complementary to the nucleic acid of interest and an enzyme capable of extending nucleic acid strands may also be present in a liquid medium containing the nucleic acid of interest introduced into the thermocycling chamber, or may be introduced into the thermocycling chamber independently of the nucleic acid of interest. In either case, each oligonucleotide of the oligonucleotide pair and the enzyme are independently bound by a photocleavable linker or a thermally cleavable linker to a bead as described herein.

As an alternative to the nucleic acid of interest being bound to a capture bead, the sample suspected of containing a nucleic acid of interest can be pre-mixed with one or more beads to which are bound at least one oligonucleotide of the oligonucleotide pair prior to being introduced into the thermocycling chamber. In this way, the nucleic acid of interest can in effect be isolated from the rest of a biological sample through pre-hybridisation to a PCR primer before being introduced into the thermocycling chamber.

Reagent beads and/or samples for amplification may be transported into a purification chamber, when used, and into the thermocycling chamber via injection pressure and capillary forces, or through the use of magnets (when the beads are magnetic), gravity or centrifugation.

In one mode of operation, the beads are mixed with a crude sample suspected of containing a target nucleic acid, which may take place in a sample purification chamber of the PCR system. The target nucleic acid attaches to the beads' nucleic acid attachment moiety (for example on a capture bead, or an oligonucleotide primer bound to a bead). The beads are then removed from the crude sample solution and transferred to a wash buffer. The wash buffer promotes removal of any species from the bead surface that may interfere with the later amplification.

The liquid medium may further comprise dNTPs and an enzyme co-factor such as MgCl₂. Generally, for a successful PCR amplification and analysis, the liquid medium, which may also be termed a test solution, comprises the four standard dNTPs, i.e. dGTP, dCTP, dATP and TTP and may also contain one or more reporter molecules that permit monitoring of the amplification by optical means as described herein. However, in some situations the reporter molecule is bound to a bead as described. A liquid medium comprising at least the four standard dNTPs, i.e. dGTP, dCTP, dATP and TTP, and any necessary enzyme cofactor may be present in the thermocycling chamber prior to the reagent beads and sample of nucleic acid being introduced.

In some examples, the test solution comprises a plurality of oligonucleotide or primer pairs, each pair complementary to a different nucleic acid of interest and each oligonucleotide of each pair bound to a bead as described. In these examples, a multiplexed PCR analysis is enabled.

In some examples, a portion of oil is provided into the thermocycling chamber. The function of the oil is to prevent evaporation of any liquid from the liquid medium during amplification. In some examples, light mineral oils such as a white oil are provided. In some examples, the volume of oil provided is in excess of the volume of the liquid medium. In some examples, sufficient oil is provided to completely envelop the liquid medium.

The method of performing PCR may include a step of confining each oligonucleotide of the oligonucleotide pair and the enzyme to a predetermined location within the thermocycling chamber prior to being released from the bead or their respective beads. This may be accomplished through the use of magnetic beads, and a series of magnets arranged within the PCR system, to manipulate the bead or beads so as to transport them to and within the thermocycling chamber. In this way, the bead or beads can be brought not only into close proximity with a heater or heater element, but also in close proximity to one another resulting in high local concentrations of the reagents at the predetermined location or locations. As a result, and once the reagents have been cleaved from the bead or their respective beads, the PCR reagents—now in solution—are in high concentrations in close proximity to the heater. This not only increases the sensitivity of the assay and the speed of amplification (due to the solution reactions occurring faster than a corresponding reaction on a solid support), but also enables a more rapid thermocycling as only that small volume of the liquid medium needs to be thermocycled, with the rest of the liquid medium acting as a heat sink to provide cooling to the small volume being thermocycled as is described later.

In some examples, the liquid medium has a volume of less than 100 μL, for example less than 50 μL, for example less than 25 μL, for example less than 10 μL, for example about 5 μL. In some examples, the liquid medium has a volume of greater than 5 μL, for example greater than 10 μL, for example greater than 25 μL, for example greater than 50 μL, for example about 100 μL. The liquid medium at the predetermined location adjacent the heater may be a small percentage (less than 1%) of the entire liquid medium in the thermocycling chamber.

The method of performing PCR includes a step of irradiating the thermocycling chamber with light of a wavelength sufficient to cleave the photocleavable linker. This step may comprise irradiating with light of a wavelength less than 400 nm. As described above, photocleavable linkers undergo photolysis at wavelengths of less than 400 nm, i.e. in the UV region of the spectrum. This step may comprise irradiating with UV-A light, i.e. light having a wavelength from 315-400 nm. Thus, a brief irradiation of the thermocycling chamber with light having a wavelength within these ranges is sufficient to cleave, for example, a nitrobenzyl photocleavable linker to release a PCR reagent in the form of an oligonucleotide primer, or a DNA elongating enzyme into the liquid medium. A suitable light source for performing the irradiation may be a mercury lamp or a UV LED. Suitable conditions have been described previously.

Oligonucleotide primers bound to beads via photocleavable linkers may not be released all at once, but can in fact be titrated in during the course of the amplification process. This can be achieved by controlling the UV flux. Ways of controlling UV flux include controlling exposure time, using neutral density filters in front of the light source, and by controlling the current of the light source (e.g. a UV LED). This controls the primer concentration in the reaction, especially in the beginning, and helps avoid primer dimerization and so non-specific amplification products.

The method may also include providing heat to the thermocycling chamber sufficient to cleave a thermally cleavable linker. A heater in thermal contact with the thermocycling chamber may provide the heat necessary to cleave a thermally cleavable linker. As described herein, a thermally cleavable linker may be an antibody, an affibody or an aptamer which specifically but non-covalently binds an oligonucleotide primer, or a DNA elongating enzyme.

Accordingly, the heat necessary to cleave a thermally cleavable linker may be accomplished by heating the thermocycling chamber comprising the liquid medium to a temperature of at least 50° C., for example at least 60° C., for example at least 70° C., for example at least 80° C., for example at least 90° C., for example up to about 95° C. Heating the thermocycling chamber comprising the liquid medium to one of these temperatures may be performed as part of a first denaturation step of a PCR amplification thermocycle, or it may be performed prior to this step.

From the moment that the beads, and all necessary components have been introduced into the thermocycling chamber, the subsequent cleavage and amplification conditions may be performed in the absence of any fluid flow within the thermocycling chamber. That is, once the beads have been localized to a predetermined location in close proximity to the heater associated with the thermocycling chamber, all fluid flow whether by stirring, external injection pressure, capillary pressure or gravity is ceased. As a result, once the oligonucleotide and enzyme reagents have been cleaved from the bead or their respective beads, these species only slowly diffuse from the bead surface to a localized region near the beads, and the rate of diffusion is greater than the rate of amplification. Thus, when the liquid medium is subjected to amplification conditions, only that localized region needs to be thermocycled.

The concentration of the nucleic acid of interest to be amplified is, at the start of the method, is greater than zero, for example greater than 10⁻²³ M (mol/l), for example greater than 10⁻²¹ M, for example greater than 10⁻²⁰ M, for example greater than 10⁻¹⁹ M. in these amounts, the amplification is sufficiently sensitive to produce an amount of amplification products that can be suitably detected.

The concentration of the nucleic acid of interest to be amplified in the PCR may be less than 1 nM, for example less than 30 pM, for example less than 1 pM, for example less than 0.1 pM, for example less than 10 fM, for example less than 1 fM, for example less than 0.1 fM.

The number of molecules of the nucleic acid of interest to be amplified in the method is, at the start of the method, preferably less than 500,000, particularly preferably less than 200,000, particularly preferably less than 100,000, particularly preferably less than 10,000. In this way, it is possible to prevent the amplification reaching saturation before its end.

In some examples, the nucleic acid of interest may be an RNA, and the method may further comprise using the RNA as a template for a reverse transcriptase to produce a cDNA strand which can then be used in a PCR amplification process. In these examples, the reverse transcriptase may also be bound to a bead using a photocleavable or thermally cleavable linker along with any necessary RNA binding primer.

In this process, and after release of the reverse transcriptase and primers from the bead or their respective beads, the liquid medium in the thermocycling chamber may be heated to a temperature in the region of 40-50° C. to allow reverse transcription to occur and produce the cDNA in situ which can then be used directly in a PCR amplification upon further heating of the liquid medium to about 95° C. to cleave a thermally cleavable linker binding a DNA polymerase to a bead.

Once the oligonucleotide pair and the enzyme have been cleaved from the bead or their respective beads into the liquid medium, the liquid medium is subjected to conditions suitable for amplification by polymerase chain reaction. The conditions suitable for amplification by polymerase chain reaction may comprise thermocycling in the absence of fluid flow within the thermocycling chamber. The conditions suitable for amplification by polymerase chain reaction may comprise heating the predetermined location to a denaturing temperature of the nucleic acid of interest by providing a pulse of energy to a micro-heater adjacent the predetermined location. The conditions suitable for amplification by polymerase chain reaction may comprise pulse-controlled amplification, in which a time varying heat flux is applied to the liquid medium, resulting in a time varying temperature gradient in the liquid medium, as will be explained in more detail below.

General amplification conditions comprise cycling a PCR mixture between three different temperatures or ranges of temperatures: typically 94-98° C. for denaturation; 50-65° C. for annealing, and 70-80° C. for chain extension or elongation, depending on polymerase.

While each of these temperatures may be considered as a step of a thermocycle, each thermocycle necessarily also involves a heating step which begins before the denaturing step, but which may also overlap with the denaturing step. In this heating step, the temperature of the liquid medium with respect to the starting temperature or the temperature of a previous elongation step is increased at least locally, i.e. at the predetermined locations where the beads and released reagents are concentrated, in order to facilitate denaturing. Subjecting the liquid medium to conditions suitable for amplification by polymerase chain reaction may therefore comprise heating the liquid medium at the predetermined location to a denaturing temperature of the nucleic acid of interest by providing a pulse of energy to a micro-heater adjacent the predetermined location.

Through the effect of the heater at least in these areas, a localised temperature of at least 90° C., for example at least 95° C., can rapidly be reached.

The length of time of heating is the total duration, in which the heater transmits heat to the predetermined location with a power suitable for denaturing of a nucleic acid sample at that predetermined location, which may correspond to a transient, localised heating of the liquid medium to a temperature of at least 90° C. If therefore the heating is produced, for example, by a flat panel heater, the heating time is the total duration, in which heat flows from the flat panel heater to the predetermined location to bring about a temperature increase at that location that is suitable for denaturing.

The unravelling of a DNA double strand and diffusion of the two strands form one another (to avoid re-hybridization), can require a sufficiently high temperature to be maintained for a certain time period of time. However, since the flat panel heater effects a localised heating, the duration of heating to effect denaturation may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

For example, for a volume of 3 μm in a microfluidic device, increasing the temperature of an aqueous solution by up by 30° C. (as would be required to take a test solution from a chain elongation temperature of 70-80° C. to a denaturation temperature of 90-95° C.) can be achieved by heating a flat panel heater in the form of a laser cut or die punched stainless steel foil with a heat flux of 4000 kW/m² for 1 ms (millisecond), with a heat flux of 400 kW/m² for 10 ms (milliseconds), or with a heat flux of 40 kW/m² for 100 ms (milliseconds). Thus, the heater is able to rapidly heat a small interfacial volume of liquid medium in the thermocycling chamber and rapidly and reliably effect denaturation.

Once denaturation has been effected, a cooling step begins before the annealing step, in order to reach the temperature required for annealing, usually 50-65° C. If the temperature in the previous denaturing step was only locally increased, the cooling may take place through heat diffusion in the liquid medium, once power to the heater has been stopped. Since the rest of the volume of the liquid medium is in comparison to the heated volume much larger, the rest of the volume serves as a heat sink to rapidly cool the heated area.

Thus, the duration of cooling to effect annealing of the primers to the nucleic acid at the predetermined location may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less than 300 μs, for example less than 100 μs, for example less than 50 μs, for example less than 30 μs, for example less than 10 μs.

Once annealing has been effected, a heating step begins before the chain extension or elongation step, in order to reach the temperature required for chain extension, usually 70-80° C.

The length of time of heating for chain extension is the total duration, in which the heater transmits heat to the predetermined location with a power suitable for chain extension of a nucleic acid sample at that predetermined location, which may correspond to a transient, localised heating of the liquid medium to a temperature of about 70-80° C. If the heating is produced, for example, by a flat panel heater, the heating time is the total duration, in which heat flows from the flat panel heater to the predetermined location to bring about a temperature increase at that location suitable for chain extension.

In one example, the annealing temperature is equal to the chain extension temperature. If the annealing temperature is equal to the chain extension temperature, only one temperature cycle with two different temperatures is required to amplify the nucleic acid of interest. The melt temperatures of the primers and the polymerase used may be selected so that at the primer melting temperature the polymerase used can still synthesize DNA at a sufficient speed. In this example, the temperature for annealing and chain extension is achieved by global heating and the denaturing step is achieved through localised heating at the predetermined location.

The duration of heating to effect chain extension may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

In one example, the heater comprises an array of 75 gold-coated tungsten wires (15 μm diameter, 200 nm Au coating) arranged on an internal surface of the thermocycling chamber. Localised heating of a layer of liquid medium of a few micrometers depth adjacent the wire is achieved via application of sub-millisecond voltage pulses to the wires. In particular, for providing localized heating for a denaturing step, a pulse at substantial peak power in the order of 1 kW for less than 500 microseconds is applied to the wire array from a 10 mF capacitator loaded to 30-40 V via a MOSFET (metal-oxide-semiconductor field-effect transistor; serving as a fast switch). This is sufficient to locally heat the solution at the predetermined location to greater than 90° C. to denature the amplicons in solution.

In another example, localized heating for a denaturing step can be achieved by heating a thin film flat panel heater (tungsten film, 15 μm thickness with a, 200 nm thick Au coating) with a heat flux of 4000 kW/m² for 1 ms (millisecond), with a heat flux of 400 kW/m² for 10 ms (milliseconds), or with a heat flux of 40 kW/m² for 100 ms (milliseconds) from a 10 mF capacitator loaded to 30-40 V via a MOSFET (metal-oxide-semiconductor field-effect transistor.

Thus, with either of these protocols, the heater is able to rapidly heat a small interfacial volume of liquid medium in the thermocycling chamber and rapidly effect chain extension of the primers by the polymerase, using a nucleic acid of interest as the template.

In some examples, the liquid medium within the thermocycling chamber is heated globally by a second heater, and maintained at a temperature of 50-60° C. (i.e., a temperature suitable for annealing). In this way, localised heating to a denaturation or chain extension temperature, and subsequent cooling, at the predetermined location can be achieved even more quickly. The second heater may be provided at a different location to the heater configured to provide localised heating. For example, the second heater may be provided in a cover layer of the system, in thermal contact with the liquid medium in the thermocycling chamber.

Through the use of flat panel or wire heaters, for example micro-heaters, and providing rapid energy pulses, it is not necessary for the whole reaction volume to be heated and that only the predetermined locations of the thermocycling chamber need be heated, i.e. those locations where the PCR reagents and nucleic acid of interest are in solution, and cooling of the micro-heaters is almost instantaneous when power to the heater is switched off. Localised heating is much more rapid than heating of the whole liquid medium as less energy needs to be transferred, and a bulk reaction (i.e., reaction in solution) is much more rapid than a surface-based reaction. Therefore, a more rapid and energy efficient PCR method can be realised.

As described herein, PCR amplification is typically monitored by fluorescence of a reporter molecule adapted for that purpose. Thus, the methods of the present disclosure may further include introducing a fluorophore into the thermocycling chamber, and detecting an optical signal by irradiating the thermocycling chamber with light of a wavelength greater than 450 nm.

An optical sensor is configured to obtain optical signals from the thermocycling chamber. In some examples, either during or at the end of each cycle of thermocycling, the optical sensor, for example a fluorescence sensor, is used to detect and measure the fluorescence level. In some examples, the fluorescence sensor detects and measures the fluorescence level after each thermocycle, or after 5 thermocycles, or after 10 thermocycles, or any number of cycles as required.

If a nucleic acid of interest is present in the sample, it will be amplified through the thermocycling, using the complementary oligonucleotide primer pair. Since the amplification of that particular nucleic acid of interest takes place in the thermocycling chamber, measurement of any presence or increase in fluorescence is an indication that the nucleic acid of interest was present in the sample or test solution. The sooner that a positive result (via fluorescence detection) confirms that a nucleic acid of interest is present in a test solution, the quicker the overall test time. The present disclosure thus enables a simple, rapid point-of-care diagnostic that can accurately and simultaneously screen for nucleic acid sequences of interest.

While the kit-of-parts, methods, systems and related aspects have been described with reference to certain examples, it will be appreciated that various modifications, changes, omissions, and substitutions can be made without departing from the spirit of the disclosure. It is intended, therefore, that kits-of-parts, methods, systems and related aspects be limited only by the scope of the following claims. Unless otherwise stated, the features of any dependent claim can be combined with the features of any of the other dependent claims, and any other independent claim.

1. A PCR kit-of-parts, comprising:
a first oligonucleotide bound to a bead by a cleavable linker;
a second oligonucleotide bound to a bead by a cleavable linker; and
an enzyme bound to a bead by a cleavable linker;
wherein
the first and second oligonucleotide form an oligonucleotide pair complementary to a nucleic acid of interest and the enzyme is capable of extending nucleic acid strands; and
each cleavable linker is independently selected from a photocleavable linker and a thermally cleavable linker.

2. The PCR kit-of-parts of claim 1, wherein the first oligonucleotide, the second oligonucleotide and the enzyme are bound to the same bead; or wherein the first oligonucleotide is bound to a first bead, the second oligonucleotide is bound to a second bead, and the enzyme is bound to a third bead.

3. The PCR kit-of-parts of claim 1, wherein one or both of the first oligonucleotide and the second oligonucleotide is bound to a bead by a photocleavable linker and/or wherein the enzyme is bound to a bead by a thermally cleavable linker.

4. The PCR kit-of-parts of claim 1, comprising a plurality of first and second oligonucleotides forming a plurality of oligonucleotide pairs, with each oligonucleotide pair being complementary to a different nucleic acid of interest, wherein each oligonucleotide of each oligonucleotide pair is bound to a bead by a cleavable linker.

5. The PCR kit-of-parts of claim 1, wherein the photocleavable linker comprises a moiety selected from an o-nitrobenzyloxy moiety; an o-nitrobenzylamino moiety; an α-substituted o-nitrobenzyl moiety, an o-nitroveratryl moiety, a phenacyl moiety, a p-alkoxyphenacyl moiety, a benzoin moiety and a pivaloyl moiety; and/or wherein the thermally cleavable linker comprises an antibody, an affibody or an aptamer specific to the enzyme.

6. A method of performing PCR, comprising:
introducing into a thermocycling chamber of a PCR system:
a sample suspected of containing a nucleic acid of interest;
an oligonucleotide pair complementary to the nucleic acid of interest; and
an enzyme capable of extending nucleic acid strands,
wherein each oligonucleotide of the oligonucleotide pair and the enzyme are independently bound by a photocleavable linker or a thermally cleavable linker to a bead dispersed in a liquid medium in the thermocycling chamber;
irradiating the thermocycling chamber with light of a wavelength sufficient to cleave the photocleavable linker and/or providing heat to the thermocycling chamber sufficient to cleave the thermally cleavable linker to release the oligonucleotide pair and the enzyme into the liquid medium;
subjecting the liquid medium to conditions suitable for amplification by polymerase chain reaction; and
detecting an optical signal from the liquid medium.

7. The method of claim 6, wherein subjecting the liquid medium to conditions suitable for amplification by polymerase chain reaction comprises thermocycling in the absence of fluid flow within the thermocycling chamber.

8. The method of claim 6, wherein irradiating the thermocycling chamber with light of a wavelength sufficient to cleave the photocleavable linker comprises irradiating with light of a wavelength less than 400 nm.

9. The method of claim 6, comprising introducing a fluorophore into the thermocycling chamber, and wherein detecting an optical signal comprises:
irradiating the thermocycling chamber with light of a wavelength greater than 450 nm.

10. The method of claim 6, comprising:
confining each oligonucleotide of the oligonucleotide pair and the enzyme
to a predetermined location within the thermocycling chamber.

11. The method of claim 10, wherein subjecting the liquid medium to conditions suitable for amplification by polymerase chain reaction comprises heating the liquid medium at the predetermined location to a denaturing temperature of the nucleic acid of interest by providing a pulse of energy to a micro-heater adjacent the predetermined location.

12. A PCR system, comprising:
a thermocycling chamber comprising a heater;
a light source configured to emit light of a wavelength less than 400 nm to the thermocycling chamber;
a light source configured to emit light of a wavelength greater than 450 nm to the thermocycling chamber; and
an optical sensor configured to obtain optical signals from the thermocycling chamber.

13. The PCR system of claim 12, wherein the heater comprises at least one micro-heater in thermal contact with a predetermined location of the thermocycling chamber.

14. The PCR system of claim 12, wherein the light source configured to emit light of a wavelength less than 400 nm comprises a mercury lamp, a laser or a UV LED.

15. The PCR system of claim 12, further comprising a computer control module operable to control, through the output of control signals, the provision of one or more pulses of electrical energy to the heater.
